(19) United States
(12) Patent Application Publication
(10) Pub. No.: US 2014/0348792 A1
(43) Pub. Date: Nov. 27, 2014

(54) METHODS TO REDUCE POLYPOSIS AND COLORECTAL CANCER

(71) Applicants: Mansour Mohamadzadeh, (US); North Carolina State University, Raleigh, NC (US)

(72) Inventors: Todd Klaenhammer, Raleigh, NC (US); Mansour Mohamadzadeh, Gainesville, FL (US)

(21) Appl. No.: 14/365,529

(22) PCT Filed: Dec. 18, 2012
(86) PCT No.: PCT/US2012/070322

§ 371 Date: Jun. 13, 2014

Related U.S. Application Data
(60) Provisional application No. 61/577,929, filed on Dec. 20, 2011.

(51) Int. Cl. A61K 35/74

(52) U.S. Cl. CPC A61K 35/747; A61K 35/741; A61K 2300/00
USPC 424/93.2

(57) ABSTRACT

Methods for reducing or inhibiting polyposis or colorectal cancer are provided. The methods comprise administering to a mammal a bacterium modified to decrease the display of lipoteichoic acid on the cell surface. Administration of the recombinant bacterium promotes a desired therapeutic response. The recombinant bacterium may be administered in a single dose or series of doses. Methods find use in treating or preventing a variety of polyp-related disorders, for example, treating or Lynch syndrome, familial adenomatous polyposis, and colorectal cancer.

### FIELD OF THE INVENTION

The present invention relates to methods and compositions for reducing or inhibiting polyposis and colorectal cancer.

### BACKGROUND OF THE INVENTION

Identifying bacterial components and endproducts that enhance protective versus pathogenic inflammation in the gut is crucial for rebalancing homeostasis in gastrointestinal (GI) chronic inflammatory diseases and malignancies. Commensal Lactobacillus species are inhabitants of the natural microbiota in the human GI tract and can stimulate innate cells to produce both inflammatory and regulatory cytokines through the interaction of their surface layer proteins. Lipoteichoic acid is a major cell wall component of lactobacilli and other lactic acid bacteria and has been reported to stimulate dendritic cells (DCs) through specific pattern recognition receptors, including TLR2, resulting in cytokine release. Disruption of LTA synthesis by deleting the phosphoglycerol transferase gene of L. acidophilus NCFM resulted in a bacterial strain (NCK2025) that acted on intestinal immune cells to augment the production of IL-10, down-regulate IL-12 levels, and significantly mitigate dextran sulfate sodium (DSS) induced and CD4+CD45RB^{high} T cell-mediated colitis in mice (PCT Application No. PCT/US2011/040674 and Mohamadzadeh, et al. PNAS (2011) 108 Supplement 1: 4623-4630). Based on these observations, LTA-deficient L. acidophilus was shown to alter the bacterial-host interaction by attenuating inflammation.

Colorectal cancer is the third most common cancer and the fourth most frequent cause of cancer deaths worldwide (Weitz et al., 2005, Lancet 365:153-65). Approximately 5-10% of all colorectal cancers are hereditary with one of the main forms being familial adenomatous polyposis (FAP), an autosomal dominant disease in which about 80% of affected individuals contain a germline mutation in the adenomatous polyposis coli (APC) gene. Further, inflammation has been shown to have a tumor-promoting role in mice with polyposis and in human colon cancer. T regulatory cells (Tregs) are potent inhibitors of inflammation and there is evidence for their protective role in polyposis and colon cancer. However, chronic interaction of Tregs with pro-inflammatory cells and their cytokines can reverse the anti-inflammatory properties of Tregs and render them pro-inflammatory. There is a consensus that interactions between lymphocytes and myeloid cells regulate pro-versus anti-tumor immunity. However, the interaction of components of the gut microflora on polyposis and associated colorectal cancer has not been clarified.

Further methods and compositions are needed in the art to improve the treatment of polyposis and reduce the occurrence of colorectal cancer.

### BRIEF SUMMARY OF THE INVENTION

Methods for reducing or inhibiting polyposis or colorectal cancer are provided. The methods comprise administering to a mammal a bacterium modified to decrease the display of lipoteichoic acid on the cell surface. Administration of the modified bacterium promotes a desired therapeutic response. The modified bacterium may be administered in a single dose or series of doses. Methods find use in treating or preventing a variety of polyp-related disorders, for example, treating or preventing Lynch syndrome, familial adenomatous polyposis, and colorectal cancer.

### BRIEF DESCRIPTION OF THE DRAWINGS

FIG. 1 reports the status of polyps in polyp-ridden mice treated with PBS, NCK56, or NCK2025. PBS, n=6. NCK56 and NCK2025, n=5. (a) Polyps were counted in the ileum and colon and results are presented using box and whisker plots. (b) Photographs of representative colons of mice treated with PBS (top) or NCK2025 (bottom). (c) Quantification of Ki-67+ and TUNEL+ cells in polyps of mice treated with PBS, NCK56 or NCK2025. (d) Stainings of colonic polyp for Ki-67 (top panels) and TUNEL (bottom panels); 6 nm sections from paraffin embedded tissue of mice treated with PBS, NCK56 or NCK2025. Bar represents 50 μm. (***p<0.0001; **p<0.004; *p<0.0176)

FIG. 2 shows the attenuation of intra-polyp inflammation in TS4Cre×cAPClox468 by L. acidophilus strains. (a) CAE+ mast cells, n=4 mice (b) Gr1+ neutrophils and ganulocytes, n=5 or more (c) F4/80+ macrophages, n=5 or more (d) frequencies intr polyp CAE+ cells, ***p<0.0001 (e) frequencies of intrapolyp Gr1+ cells, ***p<0.0005 (f) frequencies of intrapolyp F4/80+ cells, **p<0.006. Black size bar represents 50 μm, black arrows show mast cells, white arrowheads show F480+ or Gr1+ cells, For control and NCK2025 groups, n=3; for Nck56 group, n=5. SEM error bars are shown.

FIG. 3 represents the quantification of infiltration in the polyp and normal adjacent tissues by DC expressing immune suppressive and pro-inflammatory cytokines. (a) CD11c+IL-10+, *** p<0.0001, ** p<0.001 (b) CD11c+IL-12+, **p<0.006, (**)ₚ<0.004 (c) CD11c+TNFα+, ***p<0.0005, *p<0.01 (d) CD11c+ CD103+ ***p<0.0009, *p<0.05. Significant increase in intra-polyp as compared to extra-polyp CD103+ DC, **p<0.0014. White size bar represents 50 μm, white arrowheads show double positive cells.

FIG. 4 shows the change in quality of polyp-infiltrating Tregs and helper CD4 T-cells. Representations of polyp-infiltrating (a) CD4+Foxp3+ Tregs, encompassing both anti- and pro-inflammatory subsets, (b) cell expression of RORγt and/or Foxp3 encompassing anti-inflammatory and pro-inflammatory Tregs, as well as pro-inflammatory T-cells (c) CD4+IFNγ+ pro-inflammatory T-cells; mice were untreated or treated with PBS, NCK56 or NCK2025 as indicated. Quantification in the polyps and normal adjacent tissue of, (d) CD4+Foxp3+ Tregs, encompassing both anti- and pro-inflammatory subsets, *p<*0.004, (e) RORγt-Foxp3+ anti-inflammatory Treg, **p<0.002 n=5, *p<0.04 n=5, (f) RORγt+Foxp3+ pro-inflammatory Tregs, **p<0.008 n=3 PBS & 5 NCK2025 (g) RORγt+Foxp3− pro-inflammatory T-cells, trend not statistically significant, (h) CD4+IFNγ+ pro-inflammatory effector T-cells, *p<0.016 n=5. Size bar represents 50 μm, white arrowheads represents F4/80+ or Gr1+ cells.

FIG. 5 exhibits the attenuation of systemic inflammation in polyp-ridden mice by L. acidophilus strains. (a) Spleen weight in polyp-ridden mice that are untreated or treated with L. acidophilus strains; * p<0.026. (b) Frequencies of CD4+ Foxp3− effector T-cells and CD4+ Foxp3+ Treg-cells in spleen and MLN of untreated and treated polyp-ridden mice; splenic CD4+ *p<0.065, Splenic Tregs p<0.026, PBS n=3, NCK56 n=5, NCK2025 n=3. (c) Frequencies of CD11b+F4/80+ macrophages, CD11b+Gr1+ neutrophils/granulocytes, and CD11b+CD11c+ dendritic cells, amongst spleen derived MNC of untreated or treated mice; splenic CD11b+F4/80+ cells ***p<0.0001, splenic CD11b+Gr1+ cells ***p<0.0004, PBS n=3, NCK56 n=5, NCK2025 n=3. (c) Same frequencies among mesenteric lymph node (MLN) derived MNC. Histograms show compiled results with SEM; trends shown were not statistically significant. Black bars are untreated, gray are NCK56, and white bars NCK2025 treated mice.

FIG. 6 shows the impact of treatment of polyp-ridden mice by L. acidophilus strains on serum cytokine levels in the mice. Data is listed in the following order, NCK2025/NCK56/PBS. IL-6: 23.16±3.03/45.60±2.21/82.72±32.06, p=0.039 for NCK2025 compared to PBS, p=0.009 for NCK20225 compared to NCK56; IFN-g: 5.11±2.13/2.79±1.39/9.08±3.17; TNF-α: 10.33±2.66/16.79±4.02/16.19±1.77; IL-10: 18.95±95/16.07±4.89/39.14±4.31, p=0.002 for NCK2025 compared to PBS, p=0.03 for NCK56 compared to PBS; VEGF: 3.19±1.13/18.11±9.98/12.46±7.42, p=0.008 for NCK2025 compared to NCK56; G-CSF: 573.3±109.1/9361±638.8/7689±1436, p<0.0001 for NCK2025 compared to PBS and NCK56; IL-12p70: 34.15±15.46/69.76±8.60/24.64±3.50, p=0.001 for NCK56 compared to PBS; IL-22: 59.57±5.65/31.85±4.18/19.89±12.60, p=0.005 for NCK2025 compared to PBS; p values given when statistical significance was achieved. NCK2025, n=5 mice; NCK56, n=2 mice; PBS, n=3 mice; each tested in duplicate.

### DETAILED DESCRIPTION OF THE INVENTION

The present inventions now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

### Overview

Methods are provided for decreasing polyposis in a subject, including methods for treating or preventing colorectal cancer. Such methods can be employed to reduce inflammation in the gastrointestinal tract using bacteria modified to decrease the display of LTA on the bacterial cell surface. Intestinal inflammation is a contributing factor to polyposis which can later progress to colorectal cancer. Thus, the methods provided herein utilize modified bacterial strains to normalize innate and adaptive immune responses in order to prevent and treat polyposis and colorectal cancer.

### Methods of Treating or Reducing the Incidence of Polyposis

Methods are provided for treating or reducing the incidence of polyposis in a mammal comprising administration of a bacterium having been modified to decrease the display of LTA on the bacterial surface. In some embodiments, bacterial strains with decreased display of LTA can ameliorate the symptoms of established polyposis and prevent the onset of colorectal cancer. In some embodiments, methods of reducing mitotic activity within polyps, reducing intrapolyp mast cell count, and reducing the intrapolyp densities of myeloid cells are provided.

“Treating” or “reducing” or “decreasing” is herein defined as curing, healing, alleviating, relieving, altering, remedying, ameliorating, improving, or affecting the condition or the symptoms of a mammal with polyposis or colorectal cancer. Thus a decrease in polyposis can comprise, for example, at least a 5%, 10%, 20%, 30%, 40%, 50%, 60%, 70%, 80%, 90%, or 100% decrease in at least one symptom or marker of polyposis. Symptoms or markers of polyposis include, but are not limited to, intrapolyp mitotic activity, myeloid cell density (such as Gr1⁺ granulocyte), intrapolyp mast cell count, intrapolyp macrophage count (such as F4/80⁺ macrophages), intrapolyp neutrophil count, intrapolyp dendritic cell count, intrapolyp inflammation (such as pro-inflammatory cells), spleen size, spleen macrophage count, spleen granulocyte count, pro-inflammatory Treg cell count, colonic or intestinal inflammation, and number of polyps in the intestinal tract, colon or rectum. Methods to assay for the symptoms and markers of polyposis are known in the art and described elsewhere herein. The mammal to be treated can be suffering from or at risk of developing polyposis or colorectal cancer, including, for example, be suffering from or at risk of developing Lynch syndrome (hereditary nonpolyposis), familial adenomatous polyposis (FAP), MUTYH-associated polyposis, mastocytosis, splenomegaly, diabetes, or autoimmune diseases (e.g. Sjögren's Syndrome).

Administration of the LTA-deficient bacterium can be for either a prophylactic or therapeutic purpose. By “preventing” or “inhibiting” is intended that the recombinant bacterium is provided prophylactically, i.e., the bacterium is provided in advance of any symptom. The prophylactic administration of the bacterium serves to prevent or attenuate any subsequent symptom of polyposis or colorectal cancer. In some embodiments preventing or inhibiting polyposis includes a condition wherein a polyp does not form for at least 1 day, 2 days, 3 days, 4 days, 5 days, 6 days, 7 days, 10 days, 14 days, 21 days, 30 days, or more than 30 days following the first administration of a LTA-deficient bacterium. In some embodiments preventing or inhibiting polyposis includes a condition wherein a polyp does not form for at least 1 day, 2 days, 3 days, 4 days, 5 days, 6 days, 7 days, 10 days, 14 days, 21 days, 30 days, or more than 30 days following the final administration of a LTA-deficient bacterium. When provided to decrease polyposis, the bacterium is provided at (or shortly after) the onset of a symptom, or the formation of a polyp. The administration of the bacterium can thereby serve to attenuate any actual symptom, including growth or division of a polyp.

By “subject” is intended mammals. In specific embodiments, the subject mammals are primates or humans. In other embodiments, subjects include domestic mammals, such as a feline or canine, or agricultural animals, such as a ruminant, horse, swine, poultry, or sheep. In specific embodiments, the subject undergoing treatment or prevention by the bacterial strains described herein is a human. In some embodiments, the human undergoing treatment can be a newborn, infant, toddler, preadolescent, adolescent or adult. The subjects of the invention may be suffering from the symptoms of polyposis or colorectal cancer or may be at risk for developing polyposis.

### Polyposis

The methods disclosed herein relate to treatment and prevention of polyposis. As used herein, the term “polyposis” refers to a condition characterized by the presence of at least one polyp. In some embodiments, polyposis has genetic or hereditary factors, such as Lynch syndrome (hereditary nonpolyposis, HNPCC), familial adenomatous polyposis (FAP) and MUTYH-associated polyposis (MAP). In some embodiments, polyposis refers to colorectal cancer or colon cancer.

The term “polyp” or “neoplasia” refers to an abnormal growth of tissue protruding from a mucous membrane. A polyp as described herein can be a hyperplastic polyp, adenomatous polyp (adenoma), an inflammatory polyp, or any other type of polyp. As used herein, a neoplastic polyp can be a adenomatous polyp or malignant polyp. In the case of a colorectal adenoma (adenomatous polyp), the abnormal growth is confined to the mucosa. If this atypical growth extends through the muscularis mucosae, the muscle layer under the mucosa, and damages the anatomical wall, the abnormal growth is referred to as a colorectal carcinoma, or malignant polyp.

As used herein, “colorectal cancer” or “colon cancer” refers to a condition characterized by having at least one colorectal carcinoma. Colorectal cancer can include all forms of cancer of the gastrointestinal tract or colon. Colorectal cancer can include sporadic and hereditary colorectal cancers. Colorectal cancer can include malignant colon neoplasms, carcinoma in situ, typical carcinoid tumors, and atypical carcinoid tumors. Colorectal cancer can further include adenocarcinoma, squamous cell carcinoma, and adenosquamous cell carcinoma. Colorectal cancer can be associated with a hereditary syndrome selected from the group consisting of hereditary nonpolyposis colorectal cancer, familial adenomatous polyposis, Gardner's syndrome, Peutz-Jeghers syndrome, Turcot's syndrome and juvenile polyposis. As used herein colon cancer can be caused by a hereditary syndrome selected from the group consisting of hereditary nonpolyposis colorectal cancer, familial adenomatous polyposis, Gardner's syndrome, Peutz-Jeghers syndrome, Turcot's syndrome and juvenile polyposis.

In some embodiments, the polyp is found in the gastrointestinal tract or rectum. As used herein, “gastrointestinal tract” includes the antrum, duodenum, jejunum, ileum and colon.

In some embodiments, the bacteria described herein are administered to a mammal with risk factors for developing polyposis or colorectal cancer. As used herein, risk factors for developing polyposis or colorectal cancer include genetic risk factors or lifestyle risk factors. Genetic risk factors include, but are not limited to, having a mutation in a tumor suppressing gene, such as the adenomatous polyposis coli (APC) gene, or having a mutation in a mismatch repair (MMR) gene, such as the SCH2, MLH1, MSH6, and PMS2 genes. Lifestyle risk factors include, but are not limited to, elevated body mass index (BMI), obesity, low physical activity, high consumption of red and processed meat, refined grains, sweets, and alcohol, and a low consumption of fruits and vegetables.

In some embodiments, a decrease in polyposis is characterized by a decrease in intrapolyp mitotic activity. As used herein, “intrapolyp mitotic activity” refers to the division or proliferation of cells in a polyp. For example, intrapolyp mitotic activity can be measured by quantifying the population or density of Ki-67⁺ or TUNEL⁺ cells. Methods to assay for Ki-67⁺ or TUNEL⁺ cells are known in the art and described elsewhere herein.

In some embodiments, a decrease in polyposis is characterized by a decrease in intrapolyp inflammation. As used herein, “intrapolyp inflammation” refers to the inflammation of tissues within at least one polyp. A decrease in intrapolyp inflammation can be measured by identifying an increase in the levels of anti-inflammatory cytokines, or a decrease in the levels of pro-inflammatory cytokines, or any combination thereof, following administration of a LTA-deficient bacterium. In some embodiments, a decrease in intrapolyp inflammation is identified by measuring an increased ratio of anti-inflammatory Tregs to pro-inflammatory Tregs following administration of a LTA-deficient bacterium.

As used herein, the term “proinflammatory cytokine” refers to an immunoregulatory cytokine that favors inflammation. Proinflammatory cytokines of the invention include IL1-alpha, IL1-beta, TNF-alpha, IL-2, IL-3, IL-6, IL-7, IL-9, IL-12, IL-17, IL-18, TNF-alpha, LT, LIF, Oncostatin, or IFN-alpha, IFN-beta, IFN-gamma.

As used herein, the term “anti-inflammatory cytokine” refers to a naturally occurring or recombinant protein, analog thereof or fragment thereof that elicits an anti-inflammatory response in a cell that has a receptor for that cytokine. Anti-inflammatory cytokines of the invention can be immunoregulatory molecules that control the proinflammatory cytokine response. Anti-inflammatory cytokines of the invention include interleukin (IL)-1 receptor antagonist, IL-4, IL-10, IL-11, and IL-13, IL-16, IFN-alpha, TGF-beta, G-CSF.

The term, “anti-inflammatory regulatory T cells” “or anti-inflammatory Tregs” or “natural regulatory T cells” or “nTregs” or “bona fide Tregs” as used herein refers to regulatory T cells expressing anti-inflammatory cytokines, such as CD4⁺Foxp3⁺ Tregs. Anti-inflammatory Tregs can lose their anti-inflammatory properties through the expression of RORγt and loss of Foxp3, leading to conversion to TH17 cells. Alternatively, they can co-express Foxp3 and RORγt and maintain T-cell suppressive properties but gain pro-inflammatory functions. In both instances, the loss of anti-inflammatory properties of Tregs can contribute to the escalation of pathogenic inflammation and progression of polyposis. Anti-inflammatory regulatory T cells may produce anti-inflammatory cytokines, such as IL-10. The term, “pro-inflammatory regulatory T cells” as used herein refers to regulatory T cells expressing pro-inflammatory cytokines, such as Foxp3⁺ RORγt⁺ Tregs.

Therapeutically Effective Amount

By “therapeutically effective dose,” “therapeutically effective amount,” or “effective amount” is intended an amount of the LTA-deficient bacterium that, when administered to a subject, decreases polyposis, or prevents polyposis from developing, increasing or progressing. “Positive therapeutic response” refers to, for example, improving the condition of at least one of the symptoms or markers of polyposis or colorectal cancer.

An effective amount of the therapeutic agent is determined based on the intended goal. The term “unit dose” refers to a physically discrete unit suitable for use in a subject, each unit containing a predetermined quantity of the therapeutic composition calculated to produce the desired response in association with its administration, i.e., the appropriate route and treatment regimen. The quantity to be administered, both according to number of treatments and unit dose, depends on the subject to be treated, the state of the subject and the protection desired. Precise amounts of the therapeutic composition also depend on the judgment of the practitioner and are peculiar to each individual. Generally, the dosage of LTA-deficient bacteria will vary depending upon such factors as the patient's age, weight, height, sex, general medical condition and previous medical history. In specific embodiments, it may be desirable to administer the bacterium in the range of approximately 10⁴ to about 10¹² CFU, 10^{s} to 10¹¹ CFU, 10⁶ to 10¹⁰ CFU, 10⁸ to 10¹⁰ CFU or 10⁸ to 10¹² CFU per dose.

In some embodiments, the method comprises administration of multiple doses of the bacterium. The method may comprise administration of 1, 2, 3, 4, 5, 6, 7, 8, 9, 10, 15, 20, 25, 30, 35, 40, or more therapeutically effective doses of a composition comprising the bacterium as described herein. In some embodiments, doses are administered over the course of 1 day, 2 days, 3 days, 4 days, 5 days, 6 days, 7 days, 10 days, 14 days, 21 days, 30 days, or more than 30 days. In some embodiments, the method comprises continuous administration of the bacterium. The frequency and duration of administration of multiple doses of the compositions is such as to reduce or prevent an inflammatory response and thereby treat or prevent a gastrointestinal disorder. Moreover, treatment of a subject with a therapeutically effective amount of the recombinant bacterium of the invention can include a single treatment or can include a series of treatments. It will also be appreciated that the effective dosage of a bacterium used for treatment may increase or decrease over the course of a particular treatment. Changes in dosage may result and become apparent from the results of diagnostic assays for detecting polyposis known in the art and described herein.

Pharmaceutical Composition

In some embodiments, bacterial strains having a decrease in the display of LTA are administered to a subject in the form of a nutraceutical composition such as a nutritional supplement and/or food additive. In specific embodiments, the pharmaceutical or nutraceutical composition comprises a LTA-deficient bacterium that has been modified to decrease the expression of a polynucleotide or polypeptide encoding a phosphoglycerol transferase. In other embodiments, the extracts are administered to a subject in the form of a pharmaceutical composition. The administration may comprise a single dose or multiple dose administration, as described elsewhere herein.

The pharmaceutical composition may be a liquid formulation or a solid formulation. When the pharmaceutical composition is a solid formulation it may be formulated as a tablet, a sucking tablet, a chewing tablet, a chewing gum, a capsule, a sachet, a powder, a granule, a coated particle, a coated tablet, an enterocoated tablet, an enterocoated capsule, a melting strip or a film. When the pharmaceutical composition is a liquid formulation it may be formulated as an oral solution, a suspension, an emulsion or syrup. The pharmaceutical composition may be administered by the nasal, oral, vaginal, or anal routes. For anal delivery, suppositories may be used.

Suppositories may comprise binders and carriers such as polyalkalene glycols or triglycerides. The composition may further be delivered in a topical preparation or intravenous form. Said composition may further comprise a carrier material independently selected from, but not limited to, the group consisting of lactic acid fermented foods, fermented dairy products, resistant starch, dietary fibers, carbohydrates, proteins, and glycosylated proteins.

As used herein, the term “pharmaceutical composition” could be formulated as a food composition, a dietary supplement, a functional food, a medical food or a nutritional product as long as the required effect is achieved, i.e. treatment or prevention of polyposis or colorectal cancer. Said food composition may be chosen from the group consisting of beverages, yogurts, juices, ice creams, breads, biscuits, crackers, cereals, health bars, spreads and nutritional products. The food composition may further comprise a carrier material, wherein said carrier material is chosen from the group consisting of lactic acid fermented foods, fermented dairy products, non-fermented dairy products (e.g. milk) resistant starch, dietary fibers, carbohydrates, proteins and glycosylated proteins.

The pharmaceutical composition according to the invention, used according to the invention or produced according to the invention may also comprise other substances, such as an inert vehicle, or pharmaceutical acceptable adjuvants, carriers, preservatives etc., which are well known.

The present disclosure also includes combinations of the recombinant bacteria with one another, and/or with one or more other agents useful in the treatment of polyposis or colorectal cancer. For example, bacteria of the invention may be administered in combination with effective doses of conventional anti-inflammatory agents for treatment of polyposis or colorectal cancer, such as chemotherapy, FOLFOX (leucovorin [folinic acid], 5-FU, and oxaliplatin), FOLFOX (leucovorin [folinic acid], 5-FU, and oxaliplatin), CapeOX (capecitabine and oxaliplatin), bevacizumab, cetuximab, 5-FU and leucovorin, FOLFOXIRI (leucovorin, 5-FU, oxaliplatin, and irinotecan), irinotecan, with or without cetuximab, cetuximab, or panitumumab. The term “administration in combination” refers to both concurrent and sequential administration of the active agents. The combination therapies are of course not limited to the agents provided herein, but include any composition for the treatment of polyposis or colorectal cancer.

Modified Bacterial Cells with Decreased Display of LTA

The various methods of treating, decreasing, or preventing polyposis provided herein employ a modified bacterial cell with a decreased display of LTA on the cell surface. As used herein, a decrease in the display of LTA on the surface of a cell or cell surface comprises any statistically significant decrease in the level of LTA displayed on the surface of a cell, when compared to an appropriate control. Such decrease can include, for example, at least a 5%, 10%, 20%, 30%, 40%, 50%, 60%, 70%, 80%, 90%, or 100% decrease in the amount of LTA displayed on the surface of a cell. Methods to assay for the amount of LTA on the surface of a cell include, for example, butanol and hydrophobic interaction chromatography (Morath S, Geyer A, & Hartung T (2001) J Exp Med 193(3):393-397) or enzyme-linked immunosorbent assay (ELISA) (Tadler et al. (2005) J Clin Lab Anal. 1989; 3(1):21). As used herein, a “decreased display of LTA” encompasses alteration of the display of LTA on the cell surface. An alteration of the display of LTA on the cell surface is meant to encompass any structure of LTA that is different from the structure of LTA on the surface of a wild type bacterium. Thus, a “LTA-deficient” bacterium, as used herein, refers to a bacterium having an altered or decreased display of LTA on the cell surface. In some embodiments, the LTA-deficient bacterium used in the methods described herein is L. acidophilus NCK2025, described in detail in PCT Application No. PCT/US2011/040,674 and Mohamadzadeh, et al. PNAS (2011) 108 Supplement 1: 4623-4630, herein incorporated by reference in their entirety.

The term “surface,” “cell surface” or “bacterial surface,” as used herein refers to an area of the bacterial cell including and external to the plasma membrane. Gram positive bacteria contain a layer of peptidoglycan external to the plasma membrane with teichoic acids interspersed within. Gram negative bacteria further contain an outer membrane covering the peptidoglycan layer. Thus, display of the LTA on the surface according to the invention can be in or on the plasma membrane or peptidoglycan layer of Gram positive bacteria, or in or on the plasma membrane, peptidoglycan layer, or outer membrane of Gram negative bacteria.

Bacterial cells employed in the methods disclosed herein have been genetically modified to decrease the display of LTA on the cell surface. As used herein, the terms “recombinant bacterium” or “recombinant bacterial cells” refer to a bacterium or plurality or bacterial cells in which at least one genetic alteration, has been effected as to a gene of interest, or a cell which is descended from a cell so altered and which comprises the genetic alteration. Accordingly, as used herein, the term “genetically modified” or “genetic modification” refers to a genetic alteration, such as a deletion, addition or substitution, which has been effected as to a gene or nucleic acid sequence of interest. In some embodiments, the genetic alteration is an alteration caused by a mutational or recombinant technique at the hand of man. In some embodiments, the mutational technique employed by the hand of man is a selection-based mutagenesis technique, wherein the selected bacteria have a genetic modification and a decreased or altered display of LTA.

In some embodiments, a genetic alteration comprises the introduction of a heterologous polynucleotide into the genome of the bacterial cell. As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide.

Any bacteria of interest can be used in the methods described herein. In specific embodiments, the bacterium comprises a probiotic bacterium. The term “probiotic” as used herein refers to “live microorganisms, which when administered in adequate amounts confer a health benefit on a host (FAO 2001: see the website at isapp.net/docs/ProbioticDefinition.pdf) or at least one organism that contributes to the health and balance of the intestinal tract of a subject. In specific embodiments, it is also referred to as “friendly”, “beneficial”, or “good” bacteria, which when ingested by a subject assists in the maintenance of intestinal health and assists in partially or completely alleviating one or more symptoms of an illness and/or disease. As used herein, “probiotic properties” comprises enhanced gut function and stability; improved protection against infectious and non-infectious diseases; immune system modulation; alleviated lactose intolerance; improved digestion and nutrient absorption; reduced blood cholesterol; reduced allergy risk; and reduced risk of urinary tract infections. In some embodiments, probiotic properties comprise an increase in anti-inflammatory cytokine production in the subject receiving the probiotic bacterium, a decrease in pro-inflammatory cytokine production in the subject receiving the probiotic bacterium, or an increase in the ratio of anti-inflammatory to pro-inflammatory cytokine production in the subject receiving the probiotic bacterium.

In some embodiments, the bacteria described herein have been modified or selected to enhance one or more than one probiotic property. For example, in some embodiments, bacteria employed in the methods have been modified to increase adhesion to the gastrointestinal epithelium and having been further modified to decrease the display of LTA on the cell surface. In other embodiments, bacteria employed in the methods have been modified to increase resistance to acid or bile or to increase bile salt hydrolase activity and having further been modified to decrease the display of LTA on the cell surface. In other embodiments, bacteria employed in the methods have been modified to generate microbial metabolites from dietary components, for example equol or other beneficial aglycones and having further been modified to decrease the display of LTA on the cell surface. In some embodiments, bacteria employed in the methods have been modified to reduce DNA damage, or increase anti genotoxic properties against chemical carcinogens and having further been modified or selected to decrease the display of LTA on the cell surface. In some embodiments, bacteria employed in the methods have been modified or selected to increase glutathione-S-transferase, glutathione, glutathione reductase, glutathione peroxidase, superoxide dismutase, catalase, oxalate utilization, or butyrate production.

In certain embodiments, the bacteria are lactic acid bacteria. As used herein, “lactic acid bacteria” is intended bacteria from a genera selected from the following: Aerococcus, Carnobacterium, Enterococcus, Lactococcus, Lactobacillus, Leuconostoc, Oenococcus, Pediococcus, Streptococcus, Melissococcus, Alloiococcus, Dolosigranulum, Lactosphaera, Tetragenococcus, Vagococcus, and Weissella (Holzapfel et al. (2001) Am. J. Clin. Nutr. 73:365 S-373S; Sneath, ed. (1986) Bergey's Manual of Systematic Bacteriology Vol 2, Lippincott, Williams and Wilkins, Hagerstown, Md.).

In some embodiments, Lactobacillus is used. By “Lactobacillus” is meant any bacteria from the genus Lactobacillus, including but not limited to L. casei, L. paracasei, L. reuteri, L. rhamnosus, L. johnsonni, L. gasseri, L. acidophilus, L. plantarum, L. fermentum, L. salivarius, L. iners, L. bulgaricus, and numerous other species outlined by Wood et al. (Holzapfel and Wood, eds. (1995) The Genera of Lactic Acid Bacteria, Vol. 2., Springer, New York). In a specific embodiment, the bacterium is L. acidophilus NCK2025.

The production of bacteria with a decreased display of LTA, the preparation of starter cultures of such bacteria, and methods of fermenting substrates, particularly food substrates such as milk and prebiotic oligosaccharides (e.g. galacto-oligosaccharides or fructo-oligosaccharides), may be carried out in accordance with known techniques, including but not limited to those described in Mäyrä-Mäkinen and Bigret (1993) Lactic Acid Bacteria. Salminen and vonWright eds. Marcel Dekker, Inc. New York. 65-96.; Sandine (1996) Dairy Starter Cultures Cogan and Accolas eds. VCH Publishers, New York. 191-206; Gilliland (1985) Bacterial Starter Cultures for Food. CRC Press, Boca Raton, Fla. In some embodiments, the LTA-deficient bacterium is recognized as generally recognized as safe (GRAS).

Bacterial cells described herein can be cultured in suitable media, as described generally in Sambrook et al. (1989) Molecular Cloning, A Laboratory Manual (2d ed., Cold Spring Harbor Laboratory Press, Cold Spring Harbor, N.Y.).

In some embodiments, bacterial strains described herein are biologically pure cultures of a bacterium comprising at least one genetic alteration resulting in decreased display of LTA on the cell surface as described herein. In further embodiments, the bacterium comprises one or several nucleotide additions, deletions and/or substitutions. These strains may include but are not limited to: Lactobacillus acidophilus, L. gasseri, L. johnsonii, L. plantarum, Lactococcus lactis, Streptoccus thermophilus, and Enterococcus species. By “biologically pure” is intended 90%, 95%, 96%, 97%, 98%, 99%, or 100% free of other bacterial cells. In other embodiments, bacterial strains described herein are found in combination with other bacterial strains to produce mixed cultures.

A “control” or “control cell” or “control bacteria” provides a reference point for measuring changes in phenotype of the recombinant bacterial cells. A control bacteria may comprise, for example: (a) a wild-type bacterium, i.e., of the same genotype as the starting material for the genetic alteration which resulted in the subject bacterium; or (b) a bacterium of the same genotype as the starting material but which has been transformed with a null construct (i.e. with a construct which has no known effect on the trait of interest, such as a construct comprising a marker gene).

Recombinant organisms having a decrease or alteration in the display of LTA on the surface may be constructed using a variety of techniques. In the present invention, the expression of polynucleotides or polypeptides encoding at least one of the enzymes of one of the LTA assembly pathways, for example, phosphoglycerol transferase or glycosyltransferase may be decreased, as described herein.

In one embodiment, the level of an LTA-related polypeptide comprising a phosphoglycerol transferase is decreased. Phosphoglycerol transferase is a polypeptide involved the transfer of Gro-P units to a glycolipid, thereby extending the LTA chain. Various phosphoglycerol transferase polypeptides and genes encoding the polypeptides are known. As used herein, phosphoglycerol transferase encompasses LTA synthase (LtaS), glycerol phosphotransferase, glycerophosphotransferase, and any other polypeptide that catalyzes the transfer of Gro-P units for the formation of the polyglycerolphosphate backbone of LTA. Phosphoglycerol transferase is a member of the alkaline phosphatase superfamily (MdoB [COG1368] Phosphoglycerol transferase). See, for example, NCBI Accession No. NZ_ACGX01000068.1 and NC_{—}010609.1. Each of these references is herein incorporated by reference.

In another embodiment, the bacteria having altered or decreased display of LTA on the cell surface can have a decrease in the expression of glycosyltransferase. In this embodiment, the level of glycosyltransferase is decreased using any of the methods to decrease the level of a polynucleotide or polypeptide described elsewhere herein. Glycosyltransferase is a polypeptide involved in the synthesis of glycolipids and lipid anchors for LTA. The glycosyltransferase polypeptides and genes encoding the polypeptides are known. As used herein, the term glycosyltransferase refers to any polypeptide that catalyzes the synthesis of glycolipids or lipid anchors for LTA including, for example, YgpP, Ugt, BgsA, IagA, LafA, or LafB. Glycosyltransferase is a member of the Glycosyltransferase_GTB_type super family[c110013]. Various glycosyltransferases are known. See, for example, NCBI Accession No. NC_{—}010609.1 and EF138835.1. Each of these references is herein incorporated by reference.

The quality and level of D-Ala substitution on teichoic acids can decrease or alter the display of LTA on the cell surface. The synthesis of D-alanyl-LTA requires four proteins that are encoded by the dlt operon, DltA, DltB, DltC, or DltD. Thus, in some embodiments, the LTA-related polynucleotide or polypeptide can comprise the polynucleotide or polypeptide set forth in the Dlt operon, including SEQ ID NOS: 9-16. Thus, in another embodiment, the level of DltA, DltB, DltC, or DltD is decreased. Various members of the dlt operon are known. See, for example, NCBI Accession No. AAF09201 (DltA); NCBI Accession No. AAB17658.1 (DltB); NCBI Accession No. CAR86674.1 (DltC); and NCBI Accession No. CAQ65981.1 (DltD). Each of these references is herein incorporated by reference. The structure of LTA can be measured by NMR and MS using known techniques. See, for example, Morath S, Geyer A, & Hartung T (2001) J Exp Med 193(3):393-397.

Deposits

Applicant made a deposit of Lactobacillus acidophilus NCK2025 with the American Type Culture Collection (ATCC), Manassas, Va. 20110 USA, ATCC Deposit No. PTA-11587 on Jan. 10, 2011. The bacterial culture deposited with the ATCC on Jan. 10, 2011 was taken from the deposit maintained by North Carolina State University, 100 Schaub Hall, Campus Box 7624, Raleigh, N.C., 27695 since prior to the filing date of this application. Access to this deposit will be available during the pendency of the application to the Commissioner of Patents and Trademarks and persons determined by the Commissioner to be entitled thereto upon request. Upon allowance of any claims in the application, the Applicant will make the deposit available to the public pursuant to 37 C.F.R. §1.808. This deposit of Lactobacillus acidophilus NCK2025 will be maintained in the ATCC depository, which is a public depository, for a period of 30 years, or 5 years after the most recent request, or for the enforceable life of the patent, whichever is longer, and will be replaced if it becomes nonviable during that period. Additionally, Applicant has or will satisfy all of the requirements of 37 C.F.R. §§1.801-1.809, including providing an indication of the viability of the sample upon deposit. Applicant has no authority to waive any restrictions imposed by law on the transfer of biological material or its transportation in commerce.

In light of the description provided above, the following numbered embodiments are provided:

1. A method for decreasing polyposis in a mammal comprising, administering to a mammal a therapeutically effective amount of a bacterium having been genetically modified or selected to decrease the display of lipoteichoic acid (LTA) on the surface of said bacterium.

2. The method of embodiment 1, wherein said mammal has a risk factor for developing polyposis or colorectal cancer.

3. The method of embodiment 2, wherein said risk factor is a mutation in a tumor suppressor gene.

4. The method of embodiment 3, wherein said tumor suppressor gene is the adenomatous polyposis coli (APC) gene.

5. The method of any one of embodiments 1-4, wherein said mammal has at least one polyp.

6. The method of any one of embodiments 1-5, wherein said mammal has at least one polyp in the gastrointestinal tract or at least one polyp in the rectum.

7. The method of any one of embodiments 1-6, wherein said mammal has at least one neoplastic polyp.

8. The method of embodiment 7, wherein said neoplastic polyp is an adenomatous polyp.

9. The method of embodiment 8, wherein said adenomatous polyp is located in the gastrointestinal tract or located in the rectum of said mammal.

10. The method of embodiment 5 or 6, wherein said polyp is a malignant polyp.

11. The method of any one of embodiments 1-10, wherein said administration of a therapeutically effective amount of a bacterium decreases intrapolyp mitotic activity.

12. The method of embodiment 11, wherein said decrease of intrapolyp mitotic activity comprises a decrease in Ki-67+ or TUNEL+ cells.

13. The method of any one of embodiments 5-12, wherein said polyp is characterized by intrapolyp inflammation when compared to healthy neighboring tissue.

14. The method of embodiment 13, wherein said administration of a therapeutically effective amount of a bacterium decreases said intrapolyp inflammation.

15. The method of embodiment 14, wherein said decrease of intrapolyp inflammation comprises a decrease in the density of at least one cell type selected from of the group consisting of: mast cells, macrophages, F4/80+ macrophages, neutrophils, Gr1+ granulocytes, dendritic cells, and proinflammatory regulatory T cells.

17. The method of any one of embodiments 5-10 or 13-15, wherein said administration of a therapeutically effective amount of a bacterium decreases intrapolyp myeloid cell density, intrapolyp mast cell count, intrapolyp macrophage count, intrapolyp neutrophil count, intrapolyp dendritic cell count, or intrapolyp neutrophil count.

18. The method of any one of embodiments 1-17, wherein said administration of a therapeutically effective amount of a bacterium decreases spleen size, spleen macrophage count, or spleen granulocyte count.

19. A method for inhibiting polyposis in a mammal comprising, administering to a mammal a therapeutically effective amount of a bacterium having been genetically modified to decrease the display of lipoteichoic acid (LTA) on the surface of said bacterium.

20. The method of embodiment 19, wherein said mammal has a risk factor for developing polyposis or colorectal cancer.

21. The method of embodiment 20, wherein said risk factor is a mutation in a tumor suppressor gene.

22. The method of embodiment 21, wherein said tumor suppressor gene is the adenomatous polyposis coli (APC) gene.

23. The method of any one of embodiments 1-22, wherein said subject is a human.

24. The method of any one of embodiments 1-22, wherein said subject is a domestic animal.

25. The method of any one of embodiments 1-22, wherein said subject is an agricultural animal.

26. The method of any one of embodiments 1-22, wherein said bacterium is a probiotic bacterium.

27. The method of embodiment 26, wherein said probiotic bacterium is a lactic acid bacterium.

28. The method of embodiment 27, wherein said lactic acid bacterium is a Lactobacillus.

29. The method of embodiment 28, wherein said Lactobacillus is Lactobacillus acidophilus.

30. The method of embodiment 29, wherein said Lactobacillus acidophilus is Lactobacillus acidophilus NCK2025, deposited under ATCC accession number PTA-11587.

31. The method of any one of embodiments 1-30, wherein said bacterium is administered in combination with at least one other bacterial strain.

32. The method of embodiment 31, wherein said at least one other bacterial strain is a probiotic bacterium.

### EXPERIMENTAL

### Example 1

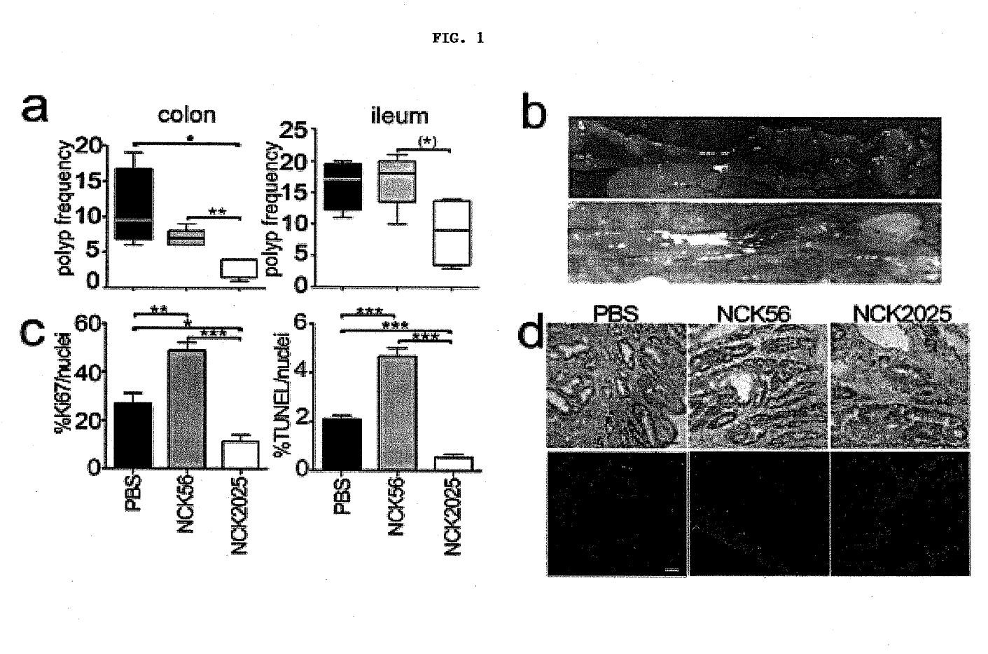

A colonic polyposis mouse model was utilized to investigate the role of the gut microbiota in the control of gastrointestinal immune balance. To analyze the immunomodulatory properties of NCK2025, polyp-ridden mice of 5 months of age were orally treated daily with doses of 5×10⁸ cfu of NCK2025, or were fed water as a control for 4 weeks. To specifically address the role of LTA, a third group of mice was treated in a similar manner with the parental L. acidophilus, NCK56. After 4 weeks of treatment, all mice were euthanized and analyzed. There was little change in polyposis in NCK56 treated mice as compared to control PBS treated mice. By contrast, NCK2025-treated mice had a reduced number of polyps in the small intestine (FIG. 1A) and significantly decreased numbers of colonic polyps (FIG. 1A, B). Mitotic and apoptotic activities were significantly reduced in polyps of NCK2025-treated mice compared to PBS- and NCK56-treated mice (FIG. 1C, D). Together, these observations demonstrated the therapeutic properties of oral NCK2025 treatment in mice with pre-established colonic polyposis and the stimulatory activity of the parental L. acidophilus strain in this model. Images were acquired using Tissue Gnostics and analyzed with Image J. The data were statistically analyzed by Unpaired T-test. Ki-67 (iHistochem) and TUNEL (Millipore) stainings were performed as per manufacturer's instructions.

### Example 2

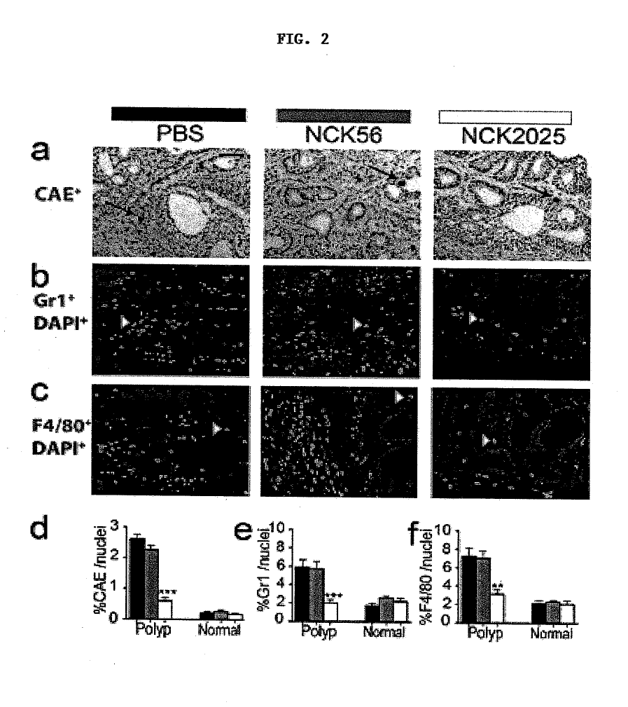

Immunofluorescent staining of paraffin sections throughout the colon was performed to provide evidence for dampened inflammation in NCK2025-treated mice. Previously, the expansion and activation of mast cells was reported in adenomatous polyps along with evidence for their tumor-promoting role (Khazaie et al. (2011) Cancer Metastasis Rev 30:45). To evaluate the impact of the gut microflora on inflammation, intrapolyp mast cell densities were quantified in mice having polyposis treated with NCK2025, compared to mice fed the parental L. acidophilus, NCK56. Significant decreases in the intrapolyp mast cell count were observed in mice fed NCK2025 reaching levels comparable to those in the colons of mice with no polyposis, but little change was found in NCK56-treated mice compared to PBS-treated mice (FIG. 2A, D). Polyps are infiltrated with relatively high densities of macrophages, neutrophils, and myeloid derived suppressor cells, therefore, quantified the impact of treatments on intrapolyp densities of F4/80⁺ macrophages and Gr1⁺ granulocytes were measured. Treatment with NCK2025 resulted in significant decreases in the intrapolyp densities of F4/80⁺ and of Gr1⁺ cells, approaching levels typical of the healthy gut (FIG. 2 B, C, E, F). Densities of these cells did not change in NCK56-treated mice. Data was analyzed by Student's t-test 2-tailed, type 2, comparing to untreated mice. Sections (5 μm thick) of frozen colon tissues were stained with Chloreacetate esterase (CAE) of specific antibodies, as described earlier (Gounaris et al., (2007) Proc Natl Acad Sci USA 104: 19977). For immunostaining primary antibodies: Rat anti-mouse F4/80 (Abcam), Biotin anti-mouse Gr1, hamster anti-mouse CD11c (BD Biosciences), anti-hamster AlexaFluor 594, anti-rat AlexaFluor 488, Streptavidin 488, and also 4,6-diamidino-2-phenylindole dihydrochloride (DAPI, Invitrogen), to reveal nuclei. Images were acquired using TissueGnostics Tissue/Cell High Throughput Imaging and Analysis System and analyzed using ImageJ software.

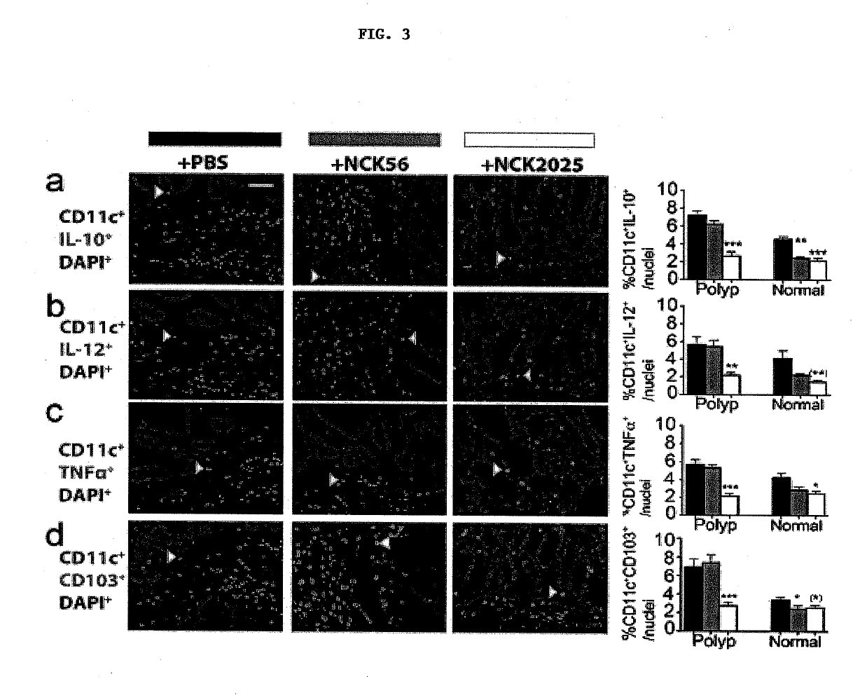

Mucosal immunity is imprinted by dendritic cells (DC). To reveal the impact of the treatments the intrapolyp expression of IL-10, IL-12 or TNF-α by residing DCs was determined. Intrapolyp DC overall tended to be higher in numbers within the polyps as compared to healthy neighboring tissue, but this difference was not statistically significant (FIG. 3 A-C), except for CD103⁺ DC which were significantly elevated within the polyps (FIG. 3D). Treatment with NCK2025 lowered the DC counts to levels approaching those of healthy wt mice, while NCK56 made little impact (FIG. 3A-D). Thus, reductions in intra-polyp densities of DC and pro-inflammatory cells suggest that NCK2025 had successfully reset the pathogenic immune environment of the gut in polyp-ridden mice back towards its physiological state. Data was analyzed by Student's t-test 2-tailed, type 2, comparing to untreated mice. Immunostaining, acquisition and analyses were done as described for FIG. 2. Antibodies: hamster anti-mouse CD11c (BD Biosciences), rat anti-mouse IL-10, IL-12, IFN-γ, TNF-α (BioLegend), anti-hamster Alexa Fluor 594, anti-rat Alexa Fluor 488.

### Example 3

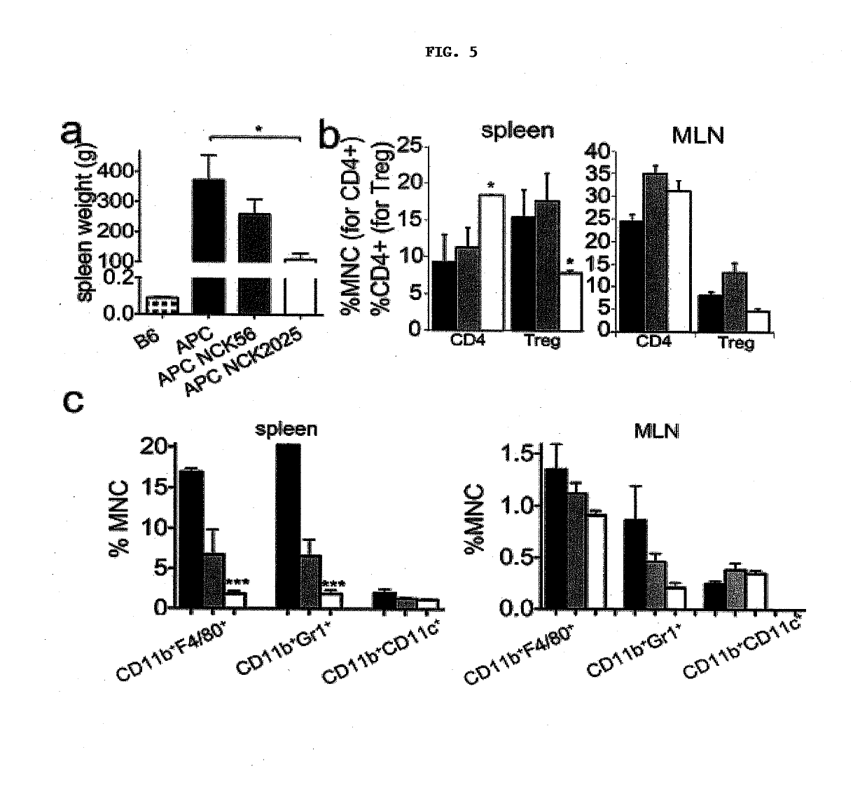

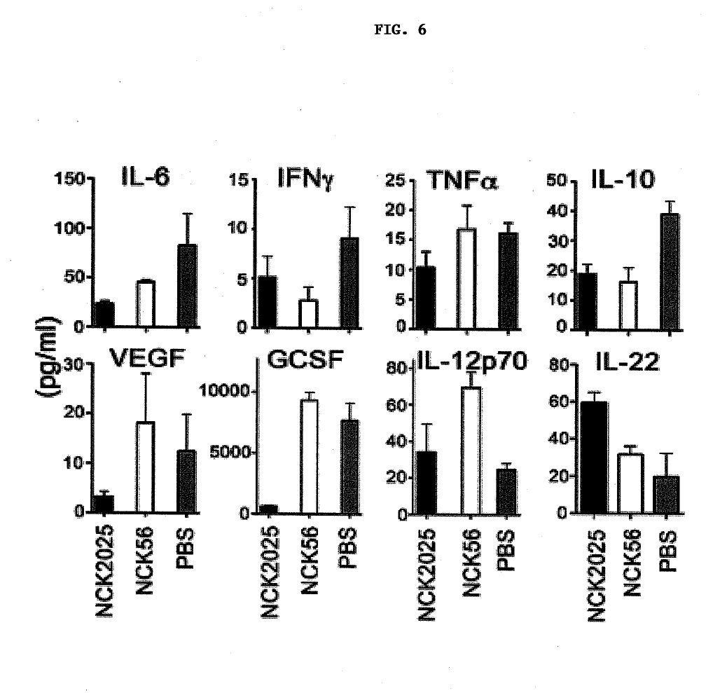

Previously we reported that inflammation associated with polyposis becomes systemic in mouse models of hereditary polyposis, which develop splenomegaly and elevated levels of serum pro-inflammatory cytokines (Gounaris et al. (2008) PLoS One 3: e2916). Aged polyp-ridden mice also developed splenomegaly (FIG. 5A). Treatment with NCK2025 caused significant reductions in spleen size while treatment with NCK56 showed similar trends, which however were not statistically significant (FIG. 5A). Reduction in spleen size correlated with increased relative frequencies of CD4 effector T-cells and reduced Tregs (FIG. 5B), and reduced macrophages and granulocyte in the spleen (FIG. 5C). Similar but not significant trends were also seen in the MLN (FIG. 5 B, C). These changes corresponded to significant drops in levels of IL-10 as well as pro-inflammatory cytokines but increase in IL-22 in the serum (FIG. 6). Single cell suspensions were filtered (40 μm), and red blood cells (RBC) were lysed using Ack Lysing Buffer (BioWhittaker). Cells were washed then incubated with Fe block (BD Bioscience). Dead cells were excluded (LIVE/DEAD Violet Dead cell Stain kit; Invitrogen). Antibodies: CD11c FITC (HL3), GR1 APC (RB6-8C5), CD4 Percp (RM4-5), CD25 biotin (7D4), and streptavidin FITC were from BD Pharmingen; F4/80 Percp (BM8) and CD11b PE-Cy7 (M1/70) were from Biolegend. Data acquired with BD FACSCanto II and analyzed using FlowJo software (Tree Star). Multiplex ELISA was conducted according to the manufactures instructions (Millipore) on filtered (0.22 mm) serum. Results were acquired with a Luminex 100 instrument and analyzed using xponent software (Luminex Corporation). Together, our findings demonstrate that administration of NCK2025 to mice with established polyps can down regulate inflammation and reset both local and systemic immunity, while the parental NCK56 strain produced an intermediate response in this model.

### Example 4

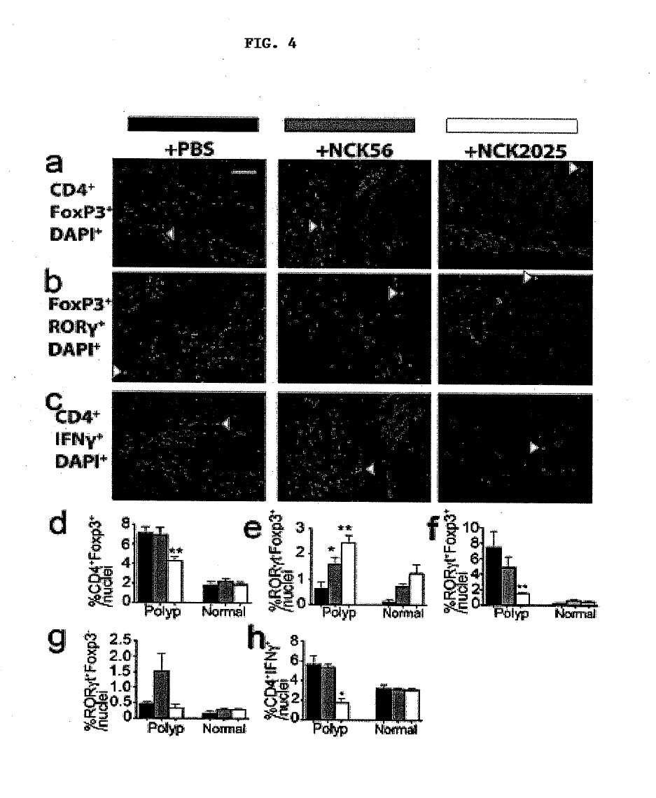

Tregs play a dual role in cancer, increasing in total numbers and suppressing protective T-cell immunity, but also protecting against cancer through suppression of inflammation. In cancer and chronic inflammation Tregs can lose their anti-inflammatory properties. This can happen through expression of RORγt and loss of Foxp3 leading to conversion to TH17 cells. Alternatively, they can co-express Foxp3 and RORγt and maintain T-cell suppressive properties but gain pro-inflammatory functions. In both instances the loss of anti-inflammatory properties of Tregs is a critical factor contribution to escalation of pathogenic inflammation (Blatner et al., (2010) Proc Natl Acad Sci US A 107: 6430). In view of the reduced inflammation in mice treated with NCK2025, we hypothesized that NCK2025 treatment contributes to the recovery of Treg anti-inflammatory properties. Immunostaining of gut tissue revealed reduced densities of Foxp3⁺ cells in polyps of NCK2025 treated mice (FIG. 4D). This does not necessarily inform of the relative ratios of pathogenic versus protective subsets of Tregs. Indeed, NCK2025 treated mice showed significant increases in densities of polyp infiltrating Foxp3⁺RORγt⁻ bona fide Tregs (FIG. 4E), and corresponding drops in densities of Foxp3⁺RORγt⁺ pro-inflammatory Tregs (FIG. 4F); mice treated with the parental NCK56 showed intermediate responses (FIG. 4E, F). Levels of RORγt expressing non-Tregs did not change with NCK2025, while pro-inflammatory CD4⁺IFNγ⁺ T-cells were reduced (FIG. 4G, H). These observations clearly show a shift in balance from pro- to anti-inflammatory Tregs in the polyp microenvironment of NCK2025-treated mice, and corresponding reduction in inflammatory helper T-cells. Our findings highlight the importance of analyzing Treg subsets separately, and implicate NCK2025 in the improvement of Treg protective functions in mice with polyposis. Data was analyzed by Student's t-test 2-tailed, type 2, comparing to untreated mice. Immunostaining, acquisition and analyses were done as described for FIG. 3. Antibodies: mouse anti-mouse CD4 (Abcam), rat anti-mouse IFN-γ (BioLegend), rat anti-mouse Foxp3 (ebiosciences), rabbit anti-mouse RoRγ, anti-rat Alexa Fluor 488, anti-mouse Alexa Flour 594, anti-rabbit Alexa Fluor 488.

Thus, pathologic inflammation in mice with pre-cancerous colonic polyps can be reset to a protective mode by oral treatment with a beneficial microbe. Healthy gut immunity is poised in a state of equilibrium that permits accurate and rapid protective responses against pathogens while curtailing pathogenic inflammatory processes. This balance is achieved through microbiota driven inflammation and IL-10 dependent suppression of inflammation by Tregs. Both pro-inflammatory and anti-inflammatory CD4⁺ T-cells are differentiated and activated through their interaction with CD11c⁺ DCs. regulatory or pro-inflammatory DCs are recognized by their expression of cytokines such as IL-10 that induce generation of extrathymice Tregs, and pro-inflammatory cytokines including IL-12 and TNF-α that commit T-cells to TH1 or TH17 lineages (36, 37). Gut-derived DCs play a significant role in imprint gut homing properties to these T-cells in a CD103 and retinoic acid-dependent manner (Mora et al., (2003) Nature 424: 88). Loss of LTA via deletion of the gene for phophoglycerol transferase, eliminates the immune stimulatory component of the interaction of L. acidophilus with the gut resulting in changes in the quality of inflammation and immunity. While densities of pro-inflammatory cells in the gut as well as the spleen were downregulated, relative densities of effector T-cells increased and Tregs decreased. These changes reflect resetting of healthy immunity in NCK2025 treated mice. An interesting outcome of this treatment was change in polyp-infiltrating Treg subsets. Cancer inflammation is regulated by Tregs, which in polyposis and cancer tend to revert to a pro-inflammatory phenotype (Gounaris et al., (2009) Cancer Res 69: 5490). In the present study, oral treatment with NCK2025 significantly increased the ratio of anti-inflammatory relative to pro-inflammatory Tregs. We expect this change to be critical for reversion from a pathogenic to a healthy level of inflammation in the gut.

That which is claimed:

1. A method for decreasing polyposis in a mammal comprising, administering to a mammal a therapeutically effective amount of a bacterium having been genetically modified to decrease the display of lipoteichoic acid (LTA) on the surface of said bacterium.

2. The method of claim 1, wherein said mammal has a risk factor for developing polyposis or colorectal cancer.

3. The method of claim 2, wherein said risk factor is a mutation in a tumor suppressor gene.

4. The method of claim 3, wherein said tumor suppressor gene is the adenomatous polyposis coli (APC) gene.

5. The method of any one of claims 1-4, wherein said mammal has at least one polyp.

6. The method of any one of claims 1-5, wherein said mammal has at least one polyp in the gastrointestinal tract or at least one polyp in the rectum.

7. The method of any one of claims 1-6, wherein said mammal has at least one neoplastic polyp.

8. The method of claim 7, wherein said neoplastic polyp is an adenomatous polyp.

9. The method of claim 8, wherein said adenomatous polyp is located in the gastrointestinal tract or located in the rectum of said mammal.

10. The method of claim 5 or 6, wherein said polyp is a malignant polyp.

11. The method of any one of claims 1-10, wherein said administration of a therapeutically effective amount of a bacterium decreases intrapolyp mitotic activity.

12. The method of claim 11, wherein said decrease of intrapolyp mitotic activity comprises a decrease in Ki-67+ or TUNEL+ cells.

13. The method of any one of claims 5-12, wherein said polyp is characterized by intrapolyp inflammation when compared to healthy neighboring tissue.

14. The method of claim 13, wherein said administration of a therapeutically effective amount of a bacterium decreases said intrapolyp inflammation.

15. The method of claim 14, wherein said decrease of intrapolyp inflammation comprises a decrease in the density of at least one cell type selected from of the group consisting of: mast cells, macrophages, F4/80+ macrophages, neutrophils. Gr1+ granulocytes, dendritic cells, and proinflammatory regulatory T cells.

16. The method of any one of claims 5-10 or 13-15, wherein said administration of a therapeutically effective amount of a bacterium decreases intrapolyp myeloid cell density, intrapolyp mast cell count, intrapolyp macrophage count, intrapolyp neutrophil count, intrapolyp dendritic cell count, or intrapolyp neutrophil count.

17. The method of any one of claims 1-16, wherein said administration of a therapeutically effective amount of a bacterium decreases spleen size, spleen macrophage count, or spleen granulocyte count.

18. A method for inhibiting polyposis in a mammal comprising, administering to a mammal a therapeutically effective amount of a bacterium having been genetically modified to decrease the display of lipoteichoic acid (LTA) on the surface of said bacterium.

19. The method of claim 18, wherein said mammal has a risk factor for developing polyposis or colorectal cancer.

20. The method of claim 19, wherein said risk factor is a mutation in a tumor suppressor gene.

21. The method of claim 20, wherein said tumor suppressor gene is the adenomatous polyposis coli (APC) gene.

22. The method of any one of claims 1-21, wherein said subject is a human.

23. The method of any one of claims 1-21, wherein said subject is a domestic animal.

24. The method of any one of claims 1-21, wherein said subject is an agricultural animal.

25. The method of any one of claims 1-21, wherein said bacterium is a probiotic bacterium.

26. The method of claim 25, wherein said probiotic bacterium is a lactic acid bacterium.

27. The method of claim 26, wherein said lactic acid bacterium is a Lactobacillus.

28. The method of claim 27, wherein said Lactobacillus is Lactobacillus acidophilus.

29. The method of claim 28, wherein said Lactobacillus acidophilus is Lactobacillus acidophilus NCK2025, deposited under ATCC accession number PTA-11587.

30. The method of any one of claims 1-29, wherein said bacterium is administered in combination with at least one other bacterial strain.

31. The method of claim 30, wherein said at least one other bacterial strain is a probiotic bacterium.
